(19) United States
(12) Patent Application Publication
(10) Pub. No.: US 2009/0082665 A1
(43) Pub. Date: Mar. 26, 2009

(54) SYSTEM AND METHOD FOR TRACKING MEDICAL DEVICE

(75) Inventor: Peter Anderson, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/861,537

(22) Filed: Sep. 26, 2007

(51) Int. Cl. A61B 5/05

(52) U.S. Cl. 600/424

(57) ABSTRACT

In one embodiment, a method of detecting a distortion is provided. The method comprises steps of identifying a change in transmitter gain for a first transmitter coil, identifying a change in transmitter gain for a second transmitter coil, comparing the change in transmitter gain for the first transmitter coil with the change in transmitter gain for the second transmitter coil and identifying a first condition upon determining that the change in transmitter gain for the first transmitter coil is different from the change in transmitter gain for the second transmitter coil.

The invention generally relates to a system and method for determining the position and orientation of a remote device relative to a reference coordinate frame using magnetic fields and more particularly to a system and method for determining the position and orientation of a medical device, such as a catheter, within a patient.

### BACKGROUND OF THE INVENTION

Many medical procedures involve a medical device, such as a drill, a catheter, scalpel, scope, stent or other tool. In some cases, a medical imaging or video system may be used to provide position information for the medical device, as well as visualization of an interior of a patient. However, medical practitioners often do not have the use of medical imaging systems when performing medical procedures. Typically, medical imaging systems are too slow to produce useable real-time images for medical device tracking in medical procedures. The use of medical imaging systems for medical device tracking may be also limited for health and safety reasons (e.g., radiation dosage concerns), financial limitations, physical space restrictions, and other concerns, for example.

Medical practitioners, such as doctors, surgeons, and other medical professionals, often rely upon technology when performing a medical procedure, such as image-guided surgery or examination. A tracking system may provide position information of the medical device with respect to the patient or a reference coordinate system, for example. A medical practitioner may refer to the tracking system to ascertain the position of the medical device when the medical device is not within the practitioner's line of sight. Moreover, the tracking system may also aid in pre surgical planning.

Thus in general, the tracking system allows the medical practitioner to visualize the patient's anatomy and track the position and orientation of the medical device. Thus, the medical practitioner may locate and operate on a desired or injured area while avoiding other structures. Increased precision in locating medical devices within the patient may provide for a less invasive medical procedure by facilitating improved control over smaller medical devices, thereby having less impact on the patient. Improved control and precision with smaller and more refined medical devices may also reduce risks associated with more invasive procedures such as open surgery.

Tracking systems may be ultrasound, inertial position, or electromagnetic tracking systems, for example. Electromagnetic tracking systems may employ coils as receivers and transmitters. Magnetic fields generated by one or more transmitter coils may be detected by one or more receiver coils. However, one disadvantage associated with the electromagnetic tracking system is, influence of metal on the measurement accuracy. Typically, an environment surrounding a medical device or a tracking system may include metal. Metal or other such substances may distort magnetic fields in the electromagnetic tracking system thereby causing the tracking system to be inaccurate.

Inaccurate position measurements produced by distortion may thus pose potential danger to the patient. Thus, a system that minimizes the effect of distortion on position measurement thereby reducing inaccurate tracking measurements would be highly desirable.

### BRIEF DESCRIPTION OF THE INVENTION

The above-mentioned needs are addressed and can be understood by reading and understanding the subject matter described herein. Various other features, medical devices, and advantages of the subject matter described herein will be made apparent to those skilled in the art from the accompanying drawings and detailed description.

In one embodiment, a tracking system comprising a transmitter assembly, a receiver assembly coupled to the transmitter assembly, a distortion detection unit and tracker electronics coupled to the receiver assembly and the distortion detection unit is provided. The transmitter assembly comprises at least one transmitter coil configured to emit a signal. The receiver assembly comprises at least one receiver coil configured for receiving the signal from the transmitter assembly. The signal represents position information of the transmitter assembly comprising at least one of position and orientation of the transmitter assembly. The distortion detection unit is configured for detecting an impact of distortion from an object on the signal and the tracker electronics is configured for determining the position of the transmitter assembly based on the signal received by the receiver assembly and the impact of distortion.

In another embodiment, a method of detecting a distortion is provided. The method comprises steps of identifying a change in transmitter gain for a first transmitter coil 505, identifying a change in transmitter gain for a second transmitter coil 510, comparing the change in transmitter gain for the first transmitter coil 505 with the change in transmitter gain for the second transmitter coil 510 and identifying a first condition upon determining that the change in transmitter gain for the first transmitter coil 505 is different from the change in transmitter gain for the second transmitter coil 510.

In yet another embodiment, a method of tracking a position of a medical device is provided. The method comprises steps of transmitting a signal from a transmitter assembly located on a medical device, receiving the signal at a receiver assembly located away from the medical device, detecting an impact of distortion on the signal and determining a position of the transmitter assembly based on the signal received by the receiver assembly and the impact of distortion.

In yet another embodiment, a method of tracking a transmitter coil is provided. The method comprises steps of obtaining a first magnetic moment of the transmitter coil, the first magnetic moment corresponding to a first predetermined time period, obtaining a second magnetic moment of the transmitter coil, the second magnetic moment corresponding to a second predetermined time period, comparing the first magnetic moment corresponding to the first predetermined time period with the second magnetic moment corresponding to the second predetermined time period, tracking a change in the magnetic moment of the transmitter coil upon detecting a difference between the first magnetic moment and the second magnetic moment and determining the presence of one of a first condition and a second condition upon tracking a change in the magnetic moment.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 is a block diagram representing an embodiment of a tracking system;

FIG. 2 is a schematic diagram representing a transmitter assembly;

FIG. 3 is a schematic diagram representing an imaging system used in conjunction with a tracking system to track a medical device in a medical object;

FIG. 4 is a flow diagram of an embodiment of a method of tracking a single transmitter coil;

FIG. 5 is a schematic diagram representing a transmitter assembly in another embodiment;

FIG. 6 is a flow diagram of an embodiment of a method of tracking a position of a medical device;

FIG. 7 is a flow diagram of an embodiment of a method of detecting a distortion; and

FIG. 8 is a flow diagram of an embodiment representing a method of identifying a change in transmitter gain.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

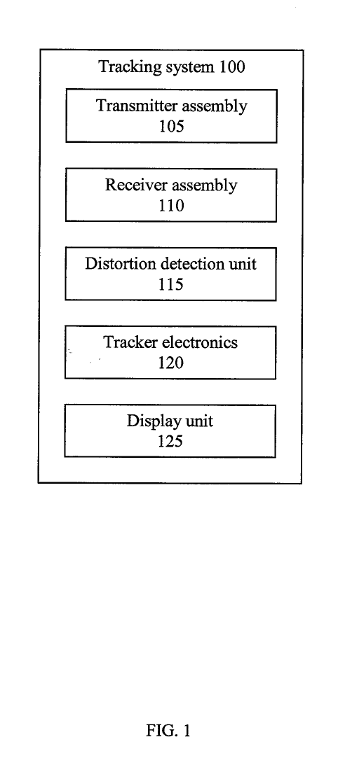

FIG. 1 illustrates a tracking system 100 provided in an embodiment of the invention. The tracking system 100 comprises a transmitter assembly 105 comprising at least one transmitter coil and configured to emit a signal, a receiver assembly 110 comprising at least one receiver coil and configured for receiving the signal from the transmitter assembly 105, a distortion detection unit 115 for detecting an impact of distortion from an object on the signal, and a tracker electronics 120 coupled to the receiver assembly 110 and the distortion detection unit 115. The tracker electronics 120 is configured for determining the position of the transmitter assembly 105 based on the signal received by the receiver assembly 110 and impact of distortion.

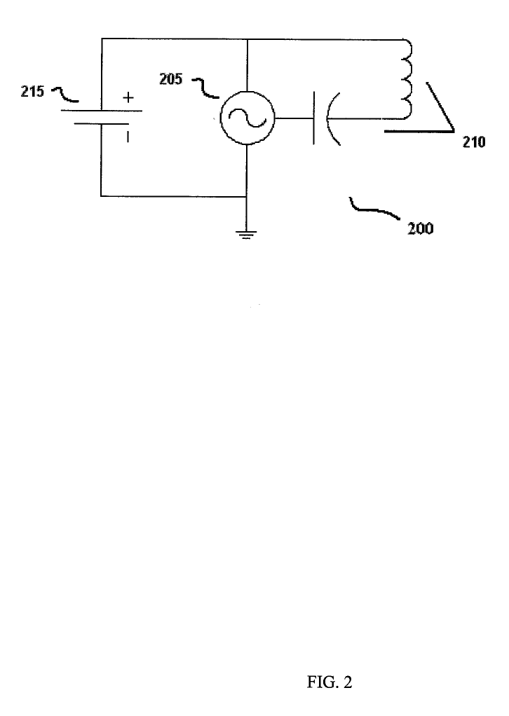

FIG. 2 shows a schematic representation of a transceiver assembly 200. The transceiver assembly 200 may be a wireless or a wired device. The transceiver assembly 200 includes an oscillator 205, which generates a carrier frequency signal in the range of about 14000 to 25000 hertz, and at least one transceiver coil 210 to which the carrier frequency signal is provided. Further, the wireless transceiver assembly 200 may have a separate power unit, such as a battery 215 or photocell, for example.

The oscillator 205 of the transceiver assembly 200 may be formed from a CMOS timer/counter circuit. The transceiver coil 210 can be one, two or three-axis magnetic sensing elements such as coil of wire, Hall-effect sensor, magnetometer and/or any sensor based on magnetoresistive, magnetoinductive, or galvanomagnetic technology. Further, the transceiver assembly 200 can be configured to function as a single coil transmitter assembly 105 or a single coil receiver assembly 110. Accordingly, the transceiver coil 210 can be configured to function as a transmitter coil or a receiver coil.

In one embodiment, the transmitter assembly 105 comprises a single magnetic field coil mounted on a medical device to be tracked. The medical device can be for example a catheter, a surgical drill or a surgical implant. The receiver assembly 110 is located remotely from the medical device and the transmitter assembly 105. The receiver assembly 110 may be affixed to a medical device guide. The medical device guide may be a drill guide or other medical device guide, for example. In general, the medical device with the medical device guide may be a tool that is indirectly controlled for applications wherein an operator's field of vision is obscured by the medical device.

In operation, the surgical drill is used to operate inside the medical object and is controlled by the drill guide. The wireless transmitter assembly 105 mounted on the surgical drill draws power from a power unit in the surgical drill and transmits a signal at a predetermined frequency. The receiver coil in the receiver assembly 110 detects the signal transmitted by the wireless transmitter coil. Using the wireless transmitter coil and the receiver assembly 110, the position of the medical device is tracked with respect to the medical device guide or other reference point, for example.

The resulting tracked position and orientation of the wireless transmitter assembly 105 attached to the surgical drill in relation to the receiver assembly 110 on the drill guide may be used to help a user manipulate the surgical drill inside the body of the medical object. This may help prevent injury to the medical object thereby minimizing unnecessary risk.

Further, each of the transmitter coil and the receiver coil is operable to generate and/or sense an electromagnetic field. Thus, the same result can alternatively be achieved with the transmitter assembly 105 replaced by the receiver assembly 110, and the receiver assembly 110 by the transmitter assembly 105. For the remainder of the description, it is assumed that the transmitter assembly 105 is mounted on the medical device to be tracked and the receiver assembly 110 is placed external to the medical object. However, the same technique can be used with the transmitter assembly 105 replaced with the receiver assembly 110 and vice versa.

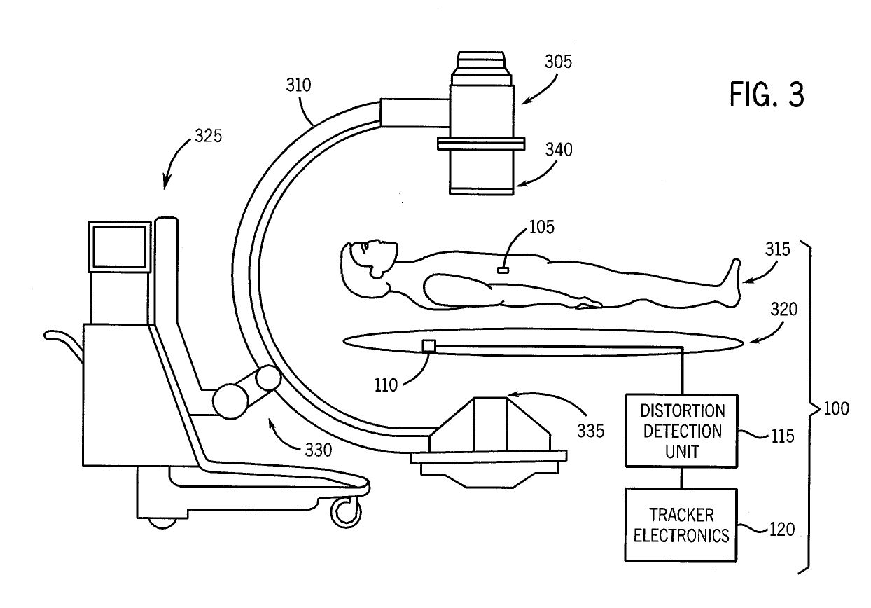

FIG. 3 shows a schematic diagram representing the electromagnetic tracking system 100 used with an image guided surgery system 305. The imaging system 305 includes a conventional C-arm 310 positioned to direct a radiation beam at a medical object 315 positioned on a patient positioning assembly 320.

The illustrated imaging system 305 comprises a main assembly 325, a mobile support assembly 330 coupled to the main assembly 325, at least one radiation source 335, and at least one radiation detector 340 configured to operate in conjunction with the radiation source 335. For mobile-type imaging systems 305, the support assembly 330 supports the radiation source 335 and/or the radiation detector 340. The main assembly 325 in combination with the support assembly 330 is operable to selectively move the radiation source 335 and the radiation detector 340 of the imaging system 305 to various positions so as to acquire image data (e.g., two-dimensional, three-dimensional) at different views of one or more regions of interest of the medical object 315.

The transmitter assembly 105 and/or the receiver assembly 110 can be coupled to one of the medical object 315, the patient positioning system 320 or a surgical implant positioned inside the medical object 315.

For the purpose of illustration only, the following detailed description references a certain embodiment of the electromagnetic tracking system 100 used with the image-guided surgery system 305. It is understood that the invention may be used with other imaging systems and other applications.

Typically methods of tracking the medical device determine the relative position and orientation of a sensing device with respect to a generator of magnetic fields, which establishes a reference coordinate frame. The sensing device here is the receiver assembly 110 and the generating device is the transmitter assembly 105.

An operator manipulates the medical device inside the medical object using the medical device guide. The wireless transmitter assembly 105 broadcasts a signal using power from the medical device. For example, the electronics of the wireless transmitter assembly 105 generate a signal using the transmitter coil of the wireless transmitter assembly 105.

The receiver coil of the receiver assembly 110 detects the signal transmitted from the wireless transmitter assembly 105. The position or orientation of the transmitter assembly 105 with respect to the medical device guide or other reference coordinate system may be determined from the received signals. In an embodiment where the receiver coil comprises multiple receiver coils, the signals are measured based on a positional relationship between the receiver coils in the receiver assembly 110.

The transmitter coil couples to the receiver coil by mutual inductance. The amount of induced coupling in the receiver coil is a function of the distance between the transmitter coil and the receiver coil and the orientation of one of the transmitter coil and the receiver coil with respect to the other. As the transmitter coil is brought into proximity with the receiver coil (at a distance of approximately 16 centimeters between the two coils), the receiver coil begins to receive the signal sent by the transmitter coil due to magnetic coupling between the two, which causes a voltage and current to be induced in the receiver coil. Thus, the amplitude of the induced voltage and current is a function of the orientation of the transmitter coil with respect to the receiver coil and the distance between the two coils.

When the longitudinal axes of the two coils are in parallel, the magnetic coupling between the two is the greatest, and the maximum current and voltage is induced in the receiver coil. When the axes of the two coils are orthogonal, the induced current and voltage in the receiver coil drops to a minimum level. Thus, the electrical current and voltage amplitude induced in the receiver coil follows a periodic rise and fall with a steady rotational motion of the transmitter coil.

The rotation of the transmitter coil amplitude modulates the 14000-hertz carrier frequency of the transmitted signal. Thus, the signal induced in the receiver coil is a 14000-hertz signal, which increases and decreases in amplitude in a periodic fashion.

The tracker electronics 120 coupled to the receiver coil is configured for processing the signal induced in the receiver coil. The tracker electronics 120 may be integrated with the receiver assembly 110 or may be a separate module, for example. In an embodiment, the tracker electronics 120 resides on the receiver assembly 110. The tracker electronics 120 may determine a ratio of mutual inductance between the transmitter coil and the receiver coil to determine the position of the transmitter coil. In order to determine the ratio of mutual inductance between the transmitter coil and the receiver coil, a ratio of currents and/or magnetic fields produced at the receiver coil may be determined.

The tracker electronics 120 includes a differential amplifier which is coupled to the receiver coil and which amplifies the received signal. The output signal of the differential amplifier is provided to a rectifier circuit, which rectifies the output signal from the differential amplifier. The rectifier circuit can be a precision half-wave rectifier comprising an operational amplifier.

The tracker electronics 120 further includes a low pass filter or integrator circuit, which receives the rectified output signal of the rectifier circuit and provides an output signal, which is, a voltage level having amplitude, which varies with the peak amplitude of the rectified output signal.

The tracker electronics 120 may also include a unity gain, buffer amplifier which is connected to the low pass filter circuit and which buffers the low pass filter circuit by providing a high impedance load to the filter circuit.

The tracking system 100 further comprises circuitry and an associated display unit 125 for indicating to a user the orientation of the medical device within the body of the medical object. The output from the buffer amplifier is provided to the display unit 125. The display unit 125 effectively translates the time relation of the amplitude peaks of the received signal into a display of the position and orientation of the transmitter coil mounted on the medical device.

The tracking system 100 may also include one or more additional transmitters for use in medical device tracking. The additional transmitter(s) may be wired or wireless transmitter(s). For example, a wireless second transmitter may be located on the medical device guide or on the medical device. The second transmitter may be wired to the tracker electronics 120 and a cable may run from the medical device to the tracker electronics 120. The wireless transmitter and additional transmitter(s) may be tracked simultaneously from the receiver coils in the receiver assembly 110. Therefore, it should also be appreciated that any number of transmitter coils can be tracked using the receiver coils.

In one embodiment, a method of locating the position of a medical device is provided. The method employs a magnetic field transmitter assembly 105 comprising a plurality of transmitter coils, which are fixed relative to one another and define a spatial reference coordinate frame, and a magnetic field receiver assembly 110 comprising one or more magnetic receiver coils. The receiver coils measure the fields produced by the transmitter coils and these measurements are then used to determine the position of the medical device relative to the reference coordinate frame.

The operating environment of the medical device may comprise a metal object, also referred to as a distorter, which may influence the induced signal in the receiver coil thereby reducing the tracking accuracy. Thus, an impact of a distorter positioned adjacent to the tracking system 100, such as adjacent to the transmitter coil or the receiver coil in the tracking system 100, may be compensated for by characterizing the tracking system 100 including the distorter. Alternatively, using a single-coil transmitter assembly 105 and receiver assembly 110, for example, allows transmitter gain to be tracked, rather than exactly characterizing the transmitter assembly 105. Such a gain tracking may help in reducing the effect of distortion in tracking the transmitter coil

For the purpose of tracking the magnetic gain, the tracking system 100 may further include a distortion detection unit 115 coupled to the tracker electronics 120. The distortion detection unit 115 may be configured for detecting and reducing the effect of distortion in tracking the transmitter coil. The configuration of the electro magnetic tracking system 100 may be adjusted using information from the distortion detection unit 115, to compensate for distortion effects from one or more metal objects present in the operating environment of the medical device.

Distortion information from the distortion detection unit 115 may be used by the tracker electronics 120 to improve the tracking accuracy. In one embodiment, the distortion detection unit 115 may include an electromagnetic field integrity detector (FID) to detect position errors induced in the tracking system 100 due to metal distortion. The field integrity detector (FID) is configured to determine a distortion field from the metal object. The distortion field may cause changes in transmitter currents, however the changes are measured and corrected by the tracker electronics 120. Thus, the use of the field integrity detector may enhace the detection of errant tracking due to magnetic field distortion introduced by the metal objects.

When tracking a single-coil wireless transmitter assembly 105, the magnetic moment of the transmitter coil is tracked simultaneously with the position and orientation of the transmitter coil. The magnetic moment is generally constant, so tracked changes in the magnetic moment indicates a plurality of conditions in the tracking system 100 and the operating environment of the medical device. The conditions include a low battery condition and a field distortion condition. The field distortion condition represents the presence of a metal distorter in the operating environment of the medical device.

In the low battery condition, the change in the current induced is proportional to changes in the battery charge. As the battery charge decreases, the current induced in the transmitter coil decreases. In the field distortion condition, the voltage induced in the transmitter coil changes in response to a distorter located within a predetermined distance from the transmitter assembly 105 in the operating environment of the medical device.

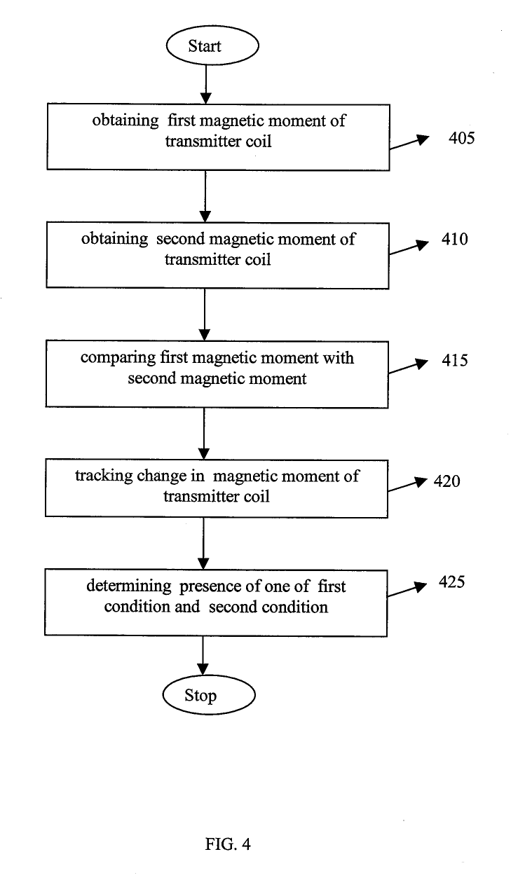

Thus in one embodiment, as shown in FIG. 4, a method of tracking a single transmitter coil is provided. The method comprises steps of obtaining a first magnetic moment of the transmitter coil step 405, the first magnetic moment corresponding to a first predetermined time period, obtaining a second magnetic moment of the transmitter coil step 410, the second magnetic moment corresponding to a second predetermined time period, comparing the first magnetic moment corresponding to the first predetermined time period with the second magnetic moment corresponding to the second predetermined time period step 415, tracking a change in the magnetic moment of the transmitter coil upon detecting a difference between the first magnetic moment and the second magnetic moment step 420 and determining the presence of one of a first condition and a second condition upon tracking a change in the magnetic moment step 425. The first condition can be the field distortion condition and the second condition can be the low battery condition.

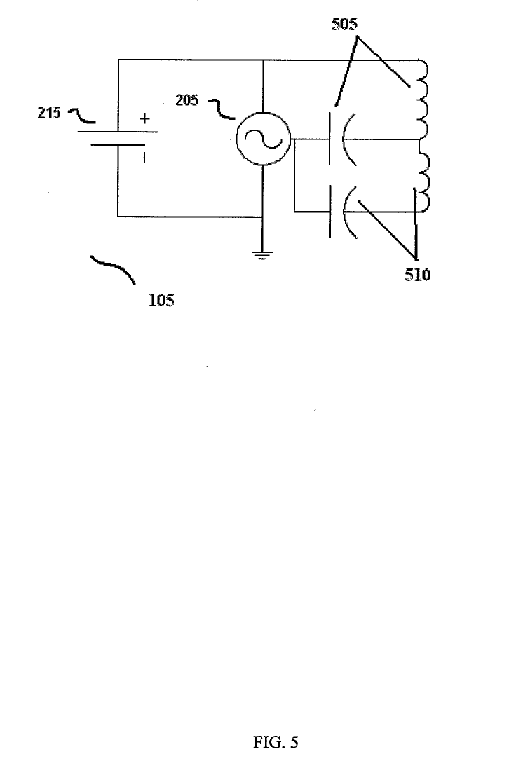

In order to distinguish between the plurality of conditions, in one embodiment as shown in FIG. 5, the transmitter assembly 105 comprises two single transmitter coils 505 and 510. Accordingly, the magnetic moments of the two-transmitter coils 505 and 510 are tracked. The magnetic moment of the two transmitter coils 505 and 510 change in unison in response to the low battery condition. Thus, in the low battery condition, the change in current induced in a first transmitter coil 505 is approximately equal to the change in current induced in a second transmitter coil 510.

As opposed to the low battery condition, the tracked magnetic moment in the first transmitter coil 505 is different from the tracked magnetic moment in the second transmitter coil 510 in the field distortion condition. Thus tracking the two transmitter coils 505 and 510 in the transmitter assembly 105 permits distinguishing the low battery condition from the field-distortion condition.

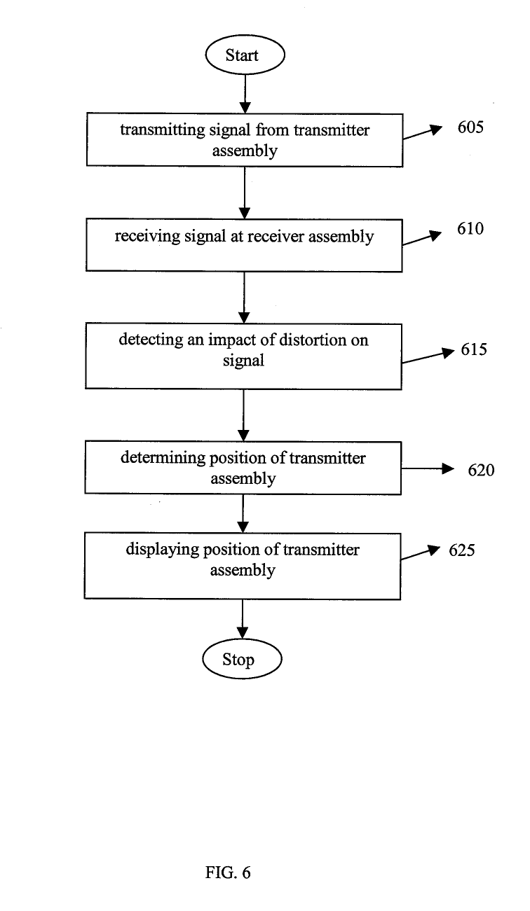

Considering the impact of distortion in tracking a position of the medical device, the method of tracking a position of the medical device includes detection of impact of distortion by the metal objects. Accordingly, FIG. 6 illustrates a flow diagram for a method for tracking a position of the medical device provided in an embodiment. The method comprises transmitting a signal from the transmitter assembly 105 located on the medical device step 605, receiving the signal at the receiver assembly 110 located away from the medical device step 610, detecting an impact of distortion on the signal step 615 and determining a position of the transmitter assembly 105 based on the signal received by the receiver assembly 110 and the impact of distortion step 620. The method may further comprise displaying the position of the transmitter assembly 105 on the display unit 125 step 625.

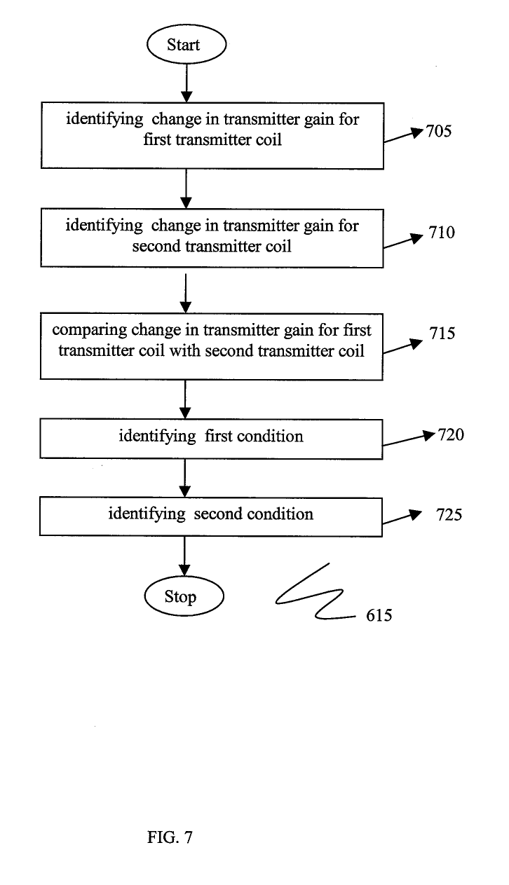

A flow diagram of the method of detecting a distortion in the tracking system 100 step 615 (FIG. 6) is illustrated in FIG. 7. At step 705, a change in transmitter gain for the first transmitter coil 505 is identified. At step 710, a change in transmitter gain for the second transmitter coil 510 is identified. At step 715, the change in transmitter gain for the first transmitter coil 505 is compared with the change in transmitter gain for the second transmitter coil 510 and at step 720, a first condition is identified upon determining that the change in transmitter gain for the first transmitter coil 505 is different from the change in transmitter gain for the second transmitter coil 510. The first condition can be a field distortion condition.

At step 725, the method 615 further comprises, identifying a second condition upon determining that the change in transmitter gain for the first transmitter coil 505 is approximately equal to the change in transmitter gain for the second transmitter coil 510. The second condition can be a low battery condition.

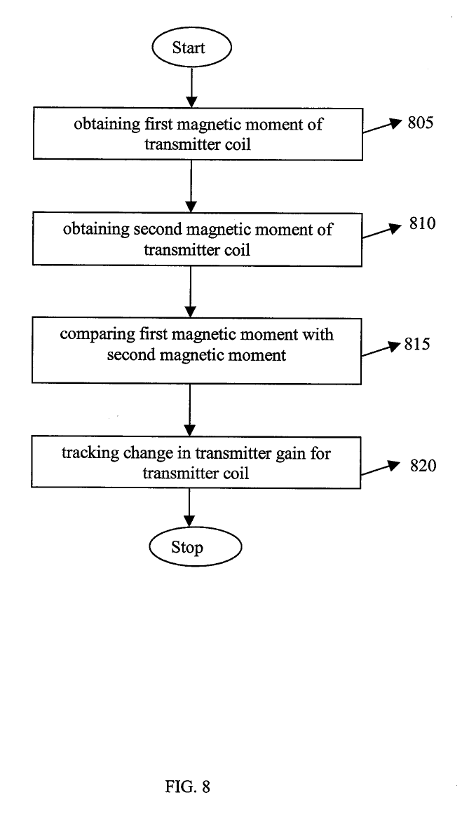

Further, a flow diagram depicting the method of identifying a change in the transmitter gain (step 705 and 710) is illustrated in FIG. 8. The method comprises steps of obtaining a first magnetic moment of a transmitter coil 505 or 510 step 805, the first magnetic moment corresponding to a first predetermined time period; obtaining a second magnetic moment of the transmitter coil 505 or 510 step 810, the second magnetic moment corresponding to a second predetermined time period, comparing the first magnetic moment corresponding to the first predetermined time period with the second magnetic moment corresponding to the second predetermined time period step 815 and tracking a change in the transmitter gain for the transmitter coil 505 or 510 upon detecting a difference between the first magnetic moment and the second magnetic moment of the transmitter coil 505 or 510 step 820.

The tracking system 100 provided in various embodiments of the invention, describes a way of determining the location and orientation of a medical device, which has been inserted into the body of the medical object. The impact of distortion on the tracking accuracy of the tracking system 100 can be measured and compensated by employing the distortion detection unit 115. Thus, the position and orientation of the medical device can be determined quickly and accurately, and the movement of the medical device in the body of the medical object may be tracked accurately.

In various embodiments, system and method for tracking a medical device are described. However, the embodiments are not limited and may be implemented in connection with different applications. The application of the invention can be extended to other areas, For example, in cardiac applications such as in catheter or flexible endoscope for tracking the path of travel of the catheter tip, to facilitate laser eye surgery by tracking the eye movements, in evaluating rehabilitation progress by measuring finger movement, to align prostheses during arthroplasty procedures and further to provide a stylus input for a Personal Digital Assistant (PDA). The invention provides a broad concept of tracking a device in obscure environment, which can be adapted to track the position of items other than medical devices in a variety of applications. That is, a tracking system may be used in other settings where the position of an instrument in an environment is unable to be accurately determined by visual inspection. For example, tracking technology may be used in forensic or security applications. Retail stores may use tracking technology to prevent theft of merchandise. Tracking systems are also often used in virtual reality systems or simulators. Accordingly, the invention is not limited to a medical device. The design can be carried further and implemented in various forms and specifications.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A tracking system comprising:
a transmitter assembly comprising at least one transmitter coil configured to emit a signal;
a receiver assembly comprising at least one receiver coil configured for receiving the signal from the transmitter assembly, the signal representing position information comprising at least one of position and orientation of the transmitter assembly;
a distortion detection unit for detecting an impact of distortion from an object on the signal; and
a tracker electronics coupled to the receiver assembly and the distortion detection unit, the tracker electronics configured for determining the position of the transmitter assembly based on the signal received by the receiver assembly and impact of distortion.

2. The tracking system of claim 1, further comprises a display for displaying the position of the transmitter assembly.

3. The tracking system of claim 2, wherein the position information is determined based on a mutual inductance between the transmitter coil and the receiver coil.

4. The tracking system of claim 2, wherein the position information is determined based on the signal and a gain ratio for the receiver assembly.

5. The tracking system of claim 3, wherein each of the transmitter coil and the receiver coil is operable to generate and/or sense an electromagnetic field.

6. The tracking system of claim 1, wherein the transmitter assembly is coupled to one of a medical object, a surgical table, a medical device or a surgical implant.

7. The tracking system of claim 1, wherein the receiver assembly is coupled to one of a medical object, a surgical table, a medical device or a surgical implant.

8. The tracking system of claim 1, wherein the receiver assembly is integrated with the tracker electronics.

9. A method of detecting a distortion, the method comprising:
identifying a change in transmitter gain for a first transmitter coil 505;
identifying a change in transmitter gain for a second transmitter coil 510;
comparing the change in transmitter gain for the first transmitter coil 505 with the change in transmitter gain for the second transmitter coil 510; and
identifying a first condition upon determining that the change in transmitter gain for the first transmitter coil 505 is different from the change in transmitter gain for the second transmitter coil 510.

10. The method of claim 9, further comprises identifying a second condition upon determining that the change in transmitter gain for the first transmitter coil 505 is approximately equal to the change in transmitter gain for the second transmitter coil 510.

11. The method of claim 9, wherein the first condition is a field distortion condition.

12. The method of claim 10, wherein the second condition is a low battery condition.

13. The method of claim 9, wherein identifying a change in the transmitter gain comprises steps of:
obtaining a first magnetic moment of a transmitter coil, the first magnetic moment corresponding to a first predetermined time period;
obtaining a second magnetic moment of the transmitter coil, the second magnetic moment corresponding to a second predetermined time period;
comparing the first magnetic moment corresponding to the first predetermined time period with the second magnetic moment corresponding to the second predetermined time period; and
tracking a change in the transmitter gain for the transmitter coil upon detecting a difference between the first magnetic moment and the second magnetic moment of the transmitter coil.

14. A method of tracking a position of a medical device, the method comprising:
transmitting a signal from a transmitter assembly located on a medical device;
receiving the signal at a receiver assembly located away from the medical device, the signal representing a position information comprising at least one of position and orientation of the transmitter assembly;
detecting an impact of distortion on the position information; and
determining a position of the transmitter assembly based on the signal received by the receiver assembly and the impact of distortion.

15. The method of claim 14, further comprising displaying the position of the transmitter assembly.

16. The method of claim 14, wherein the method of detecting a distortion comprises:
identifying a change in transmitter gain for the first transmitter coil 505;
identifying a change in transmitter gain for the second transmitter coil 510;
comparing the change in transmitter gain for the first transmitter coil 505 with the change in transmitter gain for the second transmitter coil 510; and
identifying a first condition upon determining that the change in transmitter gain for the first transmitter coil 505 is different from the change in transmitter gain for the second transmitter coil 510.

17. The method of claim 16, further comprises identifying a second condition upon determining that the change in transmitter gain for the first transmitter coil 505 is approximately equal to the change in transmitter gain for the second transmitter coil 510.

18. The method of claim 16, wherein the first condition is a field distortion condition.

19. The method of claim 17, wherein the second condition is a low battery condition.

20. The method of claim 16, wherein identifying a change in the transmitter gain comprises steps of:
obtaining a first magnetic moment of a transmitter coil, the first magnetic moment corresponding to a first predetermined time period;
obtaining a second magnetic moment of the transmitter coil, the second magnetic moment corresponding to a second predetermined time period;
comparing the first magnetic moment corresponding to the first predetermined time period with the second magnetic moment corresponding to the second predetermined time period; and
tracking a change in the transmitter gain for the transmitter coil upon detecting a difference between the first magnetic moment and the second magnetic moment.

21. A method of tracking a transmitter coil, the method comprising:
obtaining a first magnetic moment of the transmitter coil, the first magnetic moment corresponding to a first predetermined time period;
obtaining a second magnetic moment of the transmitter coil, the second magnetic moment corresponding to a second predetermined time period;
comparing the first magnetic moment corresponding to the first predetermined time period with the second magnetic moment corresponding to the second predetermined time period;
tracking a change in the magnetic moment of the transmitter coil upon detecting a difference between the first magnetic moment and the second magnetic moment; and
determining the presence of one of a first condition and a second condition upon tracking a change in the magnetic moment.

22. The method of claim 21, wherein the first condition is a field distortion condition.

23. The method of claim 21, wherein the second condition is a low battery condition.
